(19) United States
(12) Patent Application Publication
(10) Pub. No.: US 2014/0073904 A1
(43) Pub. Date: Mar. 13, 2014

(54) METHOD FOR ACQUIRING A MOVEMENT OF A PATIENT DURING A MEDICAL IMAGING EXAMINATION

(71) Applicant: Stephan Biber, Erlangen (DE)

(72) Inventor: Stephan Biber, Erlangen (DE)

(21) Appl. No.: 14/024,700

(22) Filed: Sep. 12, 2013

(30) Foreign Application Priority Data
Sep. 13, 2012 (DE) 102012216327.1

(51) Int. Cl. A61B 5/00

(52) U.S. Cl. CPC A61B 5/7292
USPC 600/410

(57) ABSTRACT

A method for acquiring a movement of a patient during a medical imaging examination, in particular a magnetic resonance examination, by means of a medical imaging device, in particular a magnetic resonance device is provided. The method includes the following method steps:
an acquisition of 3D image data by means of the medical imaging device prior to the medical imaging examination,
a calculation of 3D position information for the patient on the basis of the 3D image data,
an acquisition of movement data by means of at least one motion sensor unit during the medical imaging examination and
a determination of information regarding a movement of the patient, wherein the 3D position information for the patient and the movement data acquired by means of the at least one motion sensor unit are included in the calculation.

### CROSS REFERENCE TO RELATED APPLICATIONS

The present application claims priority to German application No. 10 2012 216327.1 DE filed Sep. 13, 2012, the entire content of which is hereby incorporated herein by reference.

### FIELD OF INVENTION

The present invention relates to a method for acquiring a movement of a patient during a medical imaging examination, in particular a magnetic resonance examination, by means of a medical imaging device, in particular a magnetic resonance device.

### BACKGROUND OF INVENTION

It is important for magnetic resonance imaging that a patient does not make any movements throughout the entire duration of the magnetic resonance examination, in particular in a region of the patient relevant to the magnetic resonance examination. Movements of the patient during the magnetic resonance examination can produce artifacts in the magnetic resonance images which can subsequently result in a misinterpretation and/or a reduction in informative value during a medical evaluation of the magnetic resonance images. It may also be necessary to repeat the magnetic resonance examination. Particularly in the case of magnetic resonance examinations of patients of a claustrophobic disposition, and/or patients suffering from pain, and/or children, it is often difficult for the patient to remain lying motionless for the duration of the magnetic resonance examination.

Methods are already known in which a movement of the body of the patient is acquired by means of the magnetic resonance examination and a modification subsequently takes place in the course of a measurement sequence, such as for example an adjustment of a gradient plane. Such methods do however need to be developed individually for each magnetic resonance sequence.

Furthermore, it is known to acquire the movement of the patient by means of a sensor unit. Thus for example a device for acquiring a movement of a patient is known from U.S. Pat. No. 8,121,361 B2 wherein marker elements, in particular optical marker elements, are arranged on the patient. A position of said marker elements is acquired by means of a detection unit and any change in position and/or movement of the patient is derived therefrom. In this situation however, prior to the magnetic resonance examination of the patient preparations for such a measurement must be undertaken by a medical operator, which results in a time-consuming preparatory phase for the magnetic resonance examination.

### SUMMARY OF INVENTION

The object of the present invention consists in particular in enabling a particularly reliable and time-saving acquisition of a movement of the patient during a magnetic resonance examination. The object is achieved by the features of the independent claims. Advantageous embodiments are described in the subclaims

The invention is based on a method for acquiring a movement of a patient during a medical imaging examination, in particular a magnetic resonance examination, by means of a medical imaging device, in particular a magnetic resonance device, comprising the following method steps:
an acquisition of 3D image data by means of the medical imaging device prior to the medical imaging examination,
a calculation of 3D position information for the patient on the basis of the 3D image data, an acquisition of movement data by means of at least one motion sensor unit during the medical imaging examination and
a determination of information regarding a movement of the patient, wherein the 3D position information for the patient and the movement data acquired by means of the at least one motion sensor unit are included in the calculation.

By this means, a movement of the patient during the magnetic resonance examination can be acquired particularly quickly and directly and it is thus also possible to react directly to the movement during the magnetic resonance examination, such as for example by modifying and/or adjusting a value of a magnetic field gradient and/or by repeating a partial measurement of the magnetic resonance examination etc. In this context, an acquisition of 3D image data is to be understood in particular to be an acquisition of 3D magnetic resonance data, in particular 3D magnetic resonance data, from an overview measurement and/or a navigator measurement by means of a magnet unit of the magnetic resonance device, in which case a three-dimensional spatial structure and/or three-dimensional positioning, in particular 3D position information, can be ascertained by means of the 3D image data for the patient arranged inside the patient receiving area. The overview measurement and/or the navigator measurement can in this situation simultaneously be formed by a measurement for slice planning and/or slice positioning of the upcoming magnetic resonance examination or by a separate measurement for acquiring 3D image data which in particular acquires an outer contour of the patient, for example a transition from tissue of the patient to air. Prior to the medical imaging examination should in particular be understood in the temporal sense. The ascertainment of the 3D position information is preferably effected by means of a central processing unit and/or a control unit, in particular a movement calculator unit, of the medical imaging device. The acquisition of the three-dimensional spatial structure and/or three-dimensional positioning of the patient advantageously takes place by means of the 3D image data on a one-off basis prior to the medical imaging examination. The movement data acquired by means of the at least one motion sensor unit is preferably formed from 1D movement data and/or from 2D movement data, in particular a 2D movement data measuring field. As a result of the combination of the medical 3D image data, in particular 3D magnetic resonance data, acquired prior to the medical imaging examination, in particular the magnetic resonance examination, and the movement data, in particular the 1D movement data and/or the 2D movement data, a particularly reliable and exact determination of a movement of the patient can take place at low cost.

It is furthermore proposed that concurrently with the acquisition of the 3D image data first movement data is acquired by means of the at least one motion sensor unit prior to the medical imaging device. In this situation a possible movement of the patient can already be acquired during the acquisition of the first 3D image data and in such a manner a particularly exact determination of the position of the patient prior to the medical imaging examination can be achieved.

It is further proposed that from the first movement data acquired prior to the medical imaging examination first information regarding a movement is ascertained which constitutes a start criterion for the medical imaging examination. Optimum and/or ideal start conditions for the medical imaging examination can be provided in this manner Particularly advantageously the start criterion for the medical imaging examination consists in the patient being motionless.

In a further embodiment of the invention it is proposed that the movement data acquired during the medical imaging examination is acquired continuously for the duration of the medical imaging examination, which means that an undesired change in position and/or movement of the patient can advantageously be acquired particularly quickly and directly for the duration of the medical imaging examination. In addition, countermeasures to at least partially compensate for the change in position and/or the movement of the patient can also be started directly hereby such that in particular artifacts can be eliminated in reconstructed magnetic resonance images.

It is furthermore proposed that the movement data acquired during the medical imaging examination and/or information calculated therefrom regarding a movement are/is stored. The acquired movement data and/or the movement information calculated therefrom can advantageously be made available for example for an evaluation, which takes place temporally delayed with respect to data acquisition, of the medical image data, in particular medical 3D image data, acquired during the medical imaging examination. The movement information can in particular during an evaluation of the medical image data acquired during the medical imaging examination result in a correction and/or modification of individual evaluation parameters.

By preference, on the basis of information regarding a movement of the patient ascertained from the movement data acquired during the medical imaging device a correction step is started which means that a correction and/or modification of measurement parameters and/or of evaluation parameters can take place on the basis of movement information ascertained. The correction step can be started for example directly after the movement information becomes available during the medical imaging examination and/or not until during an evaluation of the medical image data acquired during the medical imaging examination, in which case the evaluation step cannot take place until after the medical imaging examination. The correction step is preferably started at least partially automatically and/or in self-actuating fashion by a control unit of the medical imaging device.

Particularly advantageously, the correction step comprises a correction and/or a modification of at least one evaluation parameter during an evaluation of the medical image data from the medical imaging examination. This can be advantageous in particular in the case of for example only slight movements of the patient because the medical imaging examination can be performed in this case without interruption and a movement correction can nevertheless be taken into consideration in the evaluation of the medical image data. In particular, artifacts produced due to the movement of the patient can in this case be at least partially eliminated in the evaluation of the medical image data.

Alternatively or in addition, provision can be made that the correction step comprises a correction and/or modification of at least one measurement parameter of a measurement protocol of the medical imaging examination. Changes in position and/or movements of the patient can in this case be taken into consideration directly during the execution of the measurement protocol of the medical imaging examination. For example, a modification or correction of at least one measurement parameter of the measurement protocol of the medical imaging examination can result in a partial measurement of the medical imaging examination being aborted, in which case the partial measurement is repeated provided that the patient remains lying motionless. Furthermore, the correction and/or the modification of the at least one measurement parameter of the measurement protocol of the medical imaging examination can also include a modification and/or a correction for a magnetic field gradient such that a movement of the patient can be compensated for on the basis of the modification and/or correction of magnetic field gradient. The correction and/or modification of the at least one measurement parameter of the measurement protocol of the medical imaging examination preferably takes place during an execution of the measurement protocol of the medical imaging examination which means that current changes in position of the patient and/or movements of the patient can be taken into consideration directly and particularly quickly with regard to the acquisition of medical image data. This additionally means that it is possible to dispense with a subsequent correction of the medical image data, for example during an evaluation of the medical image data.

In a further embodiment of the invention it is proposed that information regarding a potential movement is calculated on the basis of a simulation of a movement of the patient by means of the 3D image data acquired prior to the medical imaging examination and the first movement data acquired prior to the medical imaging examination, wherein the simulation at least partially includes a model calculation and/or a fitting method. Information regarding several possible movements depending on a value of the movement data acquired can be ascertained inside the medical imaging device in such a manner on the basis of the position information acquired for the patient. It is moreover possible to back calculate particularly quickly and reliably to the movement of the patient, in which case a smaller number of different items of measurement data compared with a number of degrees of freedom of the movement of the patient to be ascertained can be incorporated into the calculation. The movement of the patient can moreover in this case be acquired and/or determined particularly cost-effectively because it is advantageously possible to dispense with a large number of motion sensor units where each degree of freedom of patient movement needs to be covered by one sensor variable.

Furthermore, it is proposed that a 3D image of the movement of the patient is simulated by means of the simulation. By preference, 2D movement data in particular is used for the simulation because here for example as a result of using a single 2D camera a particularly space-saving arrangement of the motion sensor unit inside the patient receiving area can be achieved. For example, it is possible by means of a method of least squares and/or further optimization methods to minimize a deviation of the simulated movement with the movement data acquired during the medical imaging examination, as is advantageous in particular in the case of 2D movement data.

A particularly rapid access to and/or a particularly rapid association of potential information regarding a movement, in particular calculated by means of the simulation, of the patient with movement data acquired during the medical imaging examination can be achieved if the potential movement information ascertained comprises a plurality of possible movements which are deposited in a lookup table. The lookup table is preferably deposited in a storage unit of the medical imaging device, in particular an evaluation unit of the medical imaging device. In particular, in the case of movement data which is formed from a plurality of 1D movement data items a particularly rapid association can be achieved hereby.

An association and/or a selection of one of the plurality of potential movement information items can take place by means of the movement data acquired during the medical imaging examination, which means that a number of the movements of the patient ascertained by means of the model calculation and/or the fitting method can be reduced and preferably restricted to one probable movement of the patient. To this end a plurality of movement data items acquired during the medical imaging examination, which have been acquired in temporal succession, is used in order to perform a plausibility check on the movement to be determined. For example, a movement trajectory is determined and/or calculated by means of the movement data acquired during the medical imaging examination and the model calculation and/or the fitting method, in which case the movement data acquired in temporal succession must lie on said movement trajectory.

In a further embodiment of the method according to the invention it is proposed that the at least one motion sensor unit comprises a 1D motion sensor element and/or a 2D motion sensor element, whereby a particularly cost-effective acquisition of movement data can be achieved. By preference the optical 1D motion sensor element is designed for acquisition of information regarding a movement along a single dimension and/or a single direction and the optical 2D motion sensor element is designed for acquisition of information regarding a movement along two dimensions and/or two directions, in particular a 2D sensor data measurement field. The 1D motion sensor element and/or the 2D motion sensor element can in this case be formed from conventional motion sensor elements, such as for example from an optical motion sensor element, a 2D camera, an ultrasound sensor element, a laser sensor element etc.

It is moreover proposed that the acquisition of the 3D image data acquired by means of the medical imaging device includes a navigator measurement and/or a measurement required for slice positioning. A particularly short overall examination time can be achieved hereby for the patient and the well-being of the patient thus increased by being able to acquire different items of information by means of one measurement. The navigator measurement and/or a measurement required for slice positioning preferably likewise takes place prior to the medical imaging examination on the patient. Alternatively or in addition, the 3D image data acquired by means of the medical imaging device can also be acquired by means of a separate magnetic resonance measurement. In particular, it is possible in this case to exclusively acquire data which presents information regarding a transition from a tissue to air and thus in particular acquire three-dimensional information regarding an outer contour of the patient. The separate magnetic resonance measurement preferably offers a particularly high precision in the acquisition of the outer contour of the patient which means that it is advantageously possible to dispense with further information items and thus measuring time.

Furthermore, the invention is based on a medical imaging device, in particular a magnetic resonance device, having a detector unit, a patient supporting device, a patient receiving area enclosed by the detector unit, which patient receiving area is designed to accommodate a patient positioned on the patient supporting device, and at least one motion sensor unit.

In this connection it is proposed that the medical imaging device includes a movement calculator unit, in which case the movement calculator unit is designed in order to calculate a movement of the patient on the basis of data from the detector unit and on the basis of data from the at least one motion sensor unit as claimed in the claims. By this means a movement of the patient can be determined directly during the medical imaging examination and also, if a movement of the patient occurs, the movement of the patient can advantageously be taken into consideration during the further course of the imaging examination and/or during an evaluation of the medical image data. To this end a plurality of motion sensor units is preferably arranged inside a patient receiving area and/or around a patient receiving area of the medical imaging device. Alternatively, to this end the motion sensor units can also be arranged inside a local image data acquisition unit, such as for example a local coil for a magnetic resonance examination on the patient. The motion sensor units can for example comprise a laser sensor unit and/or an ultrasound sensor unit and/or a 2D camera and/or an optical motion sensor unit etc.

It is furthermore proposed that the motion sensor unit comprises at least two motion sensor elements, wherein the at least two motion sensor elements each have a 1D motion sensor element or a 2D motion sensor element. As a result of the arrangement of a plurality of motion sensor elements for acquiring information regarding a movement of the patient it is in particular possible to reliably acquire rotational movements as well as translatory movements of the patient. By means of the at least two motion sensor elements the movement of the patient during the magnetic resonance examination can moreover be acquired in redundant fashion and/or measuring errors and/or measuring inaccuracies from the movement data acquired from the at least two motion sensor units can be eliminated.

### BRIEF DESCRIPTION OF THE DRAWINGS

Further advantages, features and details of the invention will emerge from the exemplary embodiment described in the following and with reference to the drawings.

In the drawings:

FIG. 1 shows a schematic illustration of a medical imaging device having a movement calculator unit and

FIG. 2 shows a method according to the invention for acquiring a movement of a patient.

### DETAILED DESCRIPTION OF INVENTION

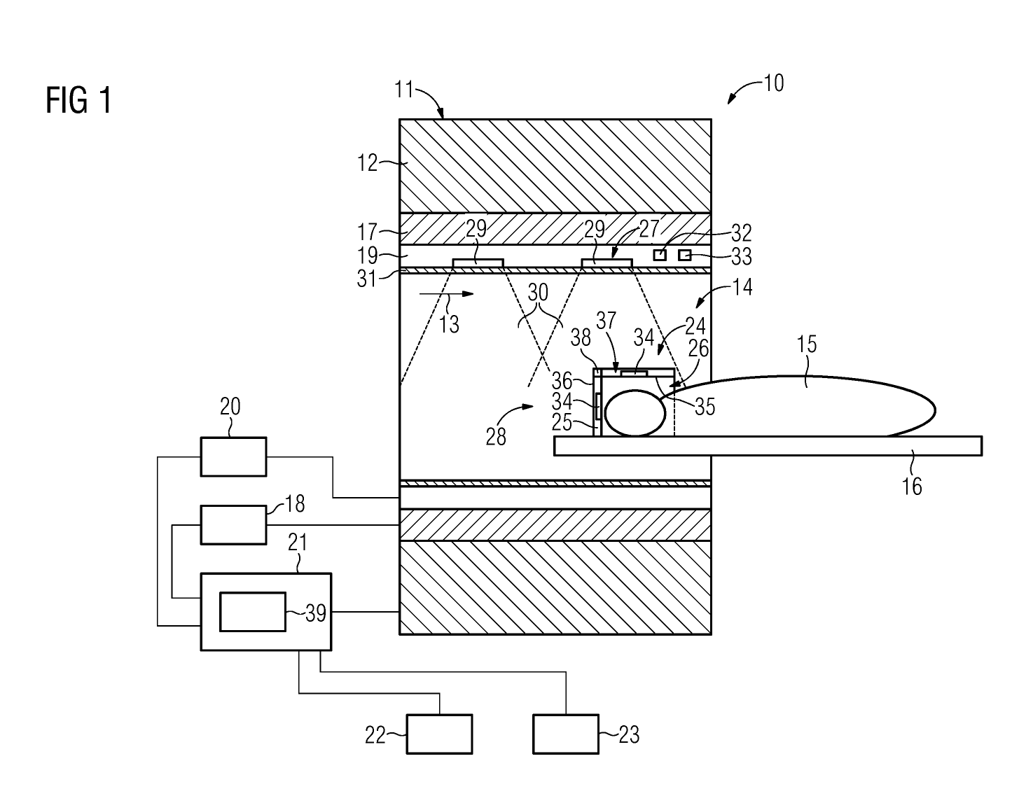

FIG. 1 illustrates a medical imaging device according to the invention which is formed by a magnetic resonance device 10. The invention is however not restricted to a development of the medical imaging device as a magnetic resonance device. Rather, the medical imaging device can also be formed by a computed tomography device and/or a PET device (positron emission tomography device) etc.

The magnetic resonance device 10 comprises a detector unit which is formed by a magnet unit 11. The magnet unit 11 comprises a primary magnet 12 for generating a strong and in particular constant primary magnetic field 13. In addition the magnetic resonance device 10 has a cylindrical patient receiving area 14 for accommodating a patient 15, where the patient receiving area 14 is enclosed in a circumferential direction by the magnet unit 11. The patient 15 can be slid into the patient receiving area 14 by means of a patient supporting device 16 of the magnetic resonance device 10.

The magnet unit 11 furthermore has a gradient coil unit 17 for generating magnetic field gradients which are used for position encoding during an imaging operation. The gradient coil unit 17 is driven by way of a gradient control unit 18. Furthermore, the magnet unit 11 has a high-frequency antenna unit 19 fixedly integrated inside the magnet unit 11 and a high-frequency antenna control unit 20 for exciting polarization which arises in the primary magnetic field 13 generated by the primary magnet 12. The high-frequency antenna unit 19 is controlled by the high-frequency antenna control unit 20 and irradiates high-frequency magnetic resonance sequences into an examination area which is essentially formed by the patient receiving area 14. By this means the magnetization is deflected from its state of equilibrium.

For controlling the primary magnet 12, the gradient control unit 18 and for controlling the high-frequency antenna control unit 20 the magnetic resonance device 10 has a system control unit 21 formed by a central processing unit. The system control unit 21 centrally controls the magnetic resonance device, such as for example the execution of a predetermined imaging magnetic field gradient echo sequence. Control information, such as for example imaging parameters, as well as reconstructed magnetic resonance images can be displayed on a display unit 22, for example a monitor, of the magnetic resonance device 10. The magnetic resonance device 10 moreover has an input unit 23 by means of which information and/or parameters can be entered by an operator during a measurement operation.

Furthermore, the magnetic resonance device 10 comprises a local magnetic resonance coil device 24 which in the present exemplary embodiment is formed by a head coil device. Alternatively, the local magnetic resonance coil device 24 can also be formed by a knee coil device, an arm coil device, a chest coil device etc. The head coil device comprises a housing unit 25 which encloses a local patient receiving area 26 of the head coil device in dome-shaped fashion.

In order to acquire a movement of the patient 15 during a magnetic resonance examination the magnetic resonance device 10 has a plurality of motion sensor units 27, 28. A first motion sensor unit 27 is enclosed by the high-frequency antenna unit 19 and a second motion sensor unit 28 is enclosed by the head coil device. Alternatively, the magnetic resonance device 10 can also include only one or more than two motion sensor units 27, 28 which are enclosed by the high-frequency antenna unit 19 and/or the local magnetic resonance coil device 24. In addition the motion sensor units 27, 28 can also at least be partially enclosed by the patient supporting device 16 and/or by further units which at least partially surround the patient receiving area 14 of the magnetic resonance device 10 and/or the local patient receiving area 26 of the local magnetic resonance coil device 24.

The motion sensor unit 27 of the high-frequency antenna unit 19 comprises a plurality of motion sensor elements 29, with only two of the motion sensor elements 29 being illustrated in FIG. 1 by way of example. The motion sensor elements 29 are in each case designed for acquiring information regarding a movement of the patient 15, with the motion sensor elements 29 to this end acquiring movement data which is formed from data not of magnetic resonance origin. The two motion sensor elements 29 each comprise an optical field of view 30 for the acquisition in each case of a partial region of the patient 15, in which case a first optical field of view 30 of a first of the two motion sensor elements 29 is directed onto a first partial region of the patient 15 or of the patient receiving area 14 and a second optical field of view 30 of a second of the two motion sensor elements 29 is directed onto a second partial region of the patient 15 or of the patient receiving area 14. The first partial region of the patient 15 or of the patient receiving area 14 and the second partial region of the patient 15 or of the patient receiving area 14 are moreover arranged at different positions on the patient 15 or inside the patient receiving area 14. The two motion sensor elements 29 thus offer a different perspective on the patient receiving area 14, in particular on the patient 15 arranged inside the patient receiving area 14, for the acquisition of information regarding a movement of the patient 15.

The high-frequency antenna unit 19 comprises a housing unit 31 which surrounds the patient receiving area 14 of the magnetic resonance device 10 in cylindrical fashion. The two motion sensor elements 29 are arranged on the housing unit 31, wherein the two motion sensor elements 29 are arranged in a region of the high-frequency antenna unit 19 which is arranged on a side of the housing unit 31 facing away from the patient receiving area 14. The two motion sensor elements 29 are thus arranged outside the patient receiving area 14.

In addition, the two motion sensor elements 29 are each formed by an optical 1D motion sensor element and/or an optical 2D motion sensor element. Information regarding a movement of the patient 15 inside the patient receiving area 14 is acquired by means of the optical 1D motion sensor elements and/or the optical 2D motion sensor elements along one dimension and/or along two dimensions, in particular a 2D sensor data measurement field. The 1D motion sensor elements can for example comprise an optical motion sensor element, an ultrasound sensor element, a laser sensor element etc. The 2D motion sensor elements can for example comprise a 2D camera etc.

The motion sensor unit 27 of the high-frequency antenna unit 19 has an evaluation unit 32 and a data transfer unit 33. The data transfer unit comprises a data send unit having an antenna element (not shown in detail) for cable-free and/or wireless data transfer. The evaluated sensor data and/or the sensor data received from the motion sensor unit 27 is transferred to a data transfer unit (not shown in detail) of the system control unit 21 by means of the data transfer unit 33.

The motion sensor unit 28 of the head coil device likewise comprises a plurality of motion sensor elements 34. Said motion sensor elements 34 are likewise formed by 1D motion sensor elements and/or by 2D motion sensor elements. The 1D motion sensor elements can for example comprise an optical motion sensor element, an ultrasound sensor element, a laser sensor element etc. The 2D motion sensor elements can for example comprise a 2D camera etc.

A housing unit of the head coil device comprises an inner housing unit 35 and an outer housing unit 36, wherein a closed space 37 is present in the head coil device between the inner housing unit 35 and the outer housing unit 36, in which closed space 37 are arranged high-frequency coil elements of the head coil unit for the acquisition of magnetic resonance signals. The motion sensor elements 34 are arranged between the inner housing unit 35 and the outer housing unit 36, wherein the motion sensor elements 34 have an angle of view different in each case from the other motion sensor elements 34 onto the patient 14 and/or the local patient receiving area 26 of the head coil device, which means that a position of the patient 14 inside the local patient receiving area 26 of the head coil device can be acquired from different perspectives.

The motion sensor unit 28 of the head coil device has a data transfer unit 38. The data transfer unit 38 comprises a data send unit having an antenna element (not shown in detail) for a cable-free and/or wireless data transfer. The evaluated sensor data and/or the sensor data received from the motion sensor elements 34 is transferred to a data transfer unit of the system control unit 21 by means of the data transfer unit 38.

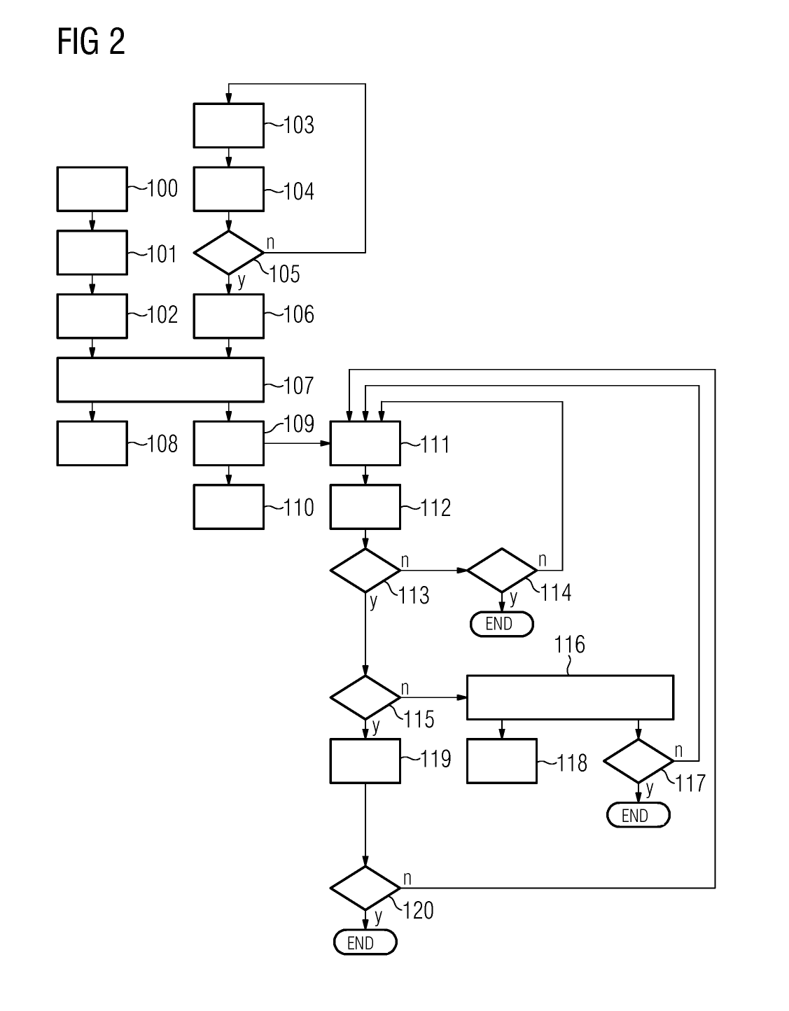

In order to determine a movement of the patient 14, a method according to the invention for determining a movement of the patient 14 during a magnetic resonance examination is carried out by means of the system control unit 21. Said method for determining the movement of the patient 14 is illustrated in detail in FIG. 2. The system control unit 21 is designed specifically for carrying out the method for determining a movement of the patient 14 during the magnetic resonance examination, wherein to this end the system control unit 21 comprises special computer programs and special software units which are stored in a storage unit (not shown in detail) of the system control unit 21 and are executed on a processor (not shown in detail) of the system control unit 21. In addition, to this end the system control unit 21 has a movement calculator unit 39.

In said method, firstly in a first method step 100 first 3D image data is acquired by means of a first magnetic resonance measurement by means of magnet unit 11, wherein the first magnetic resonance measurement is performed prior to the magnetic resonance examination on the patient 15. Said first magnetic resonance measurement is formed from an overview measurement, for example from a navigator measurement and/or further measurements required for slice positioning. To this end the system control unit 21 has evaluation software which is specifically designed for determining an outer contour of the patient 15 on the basis of the acquired 3D image data of the overview measurement. In method step 100 the 3D image data can moreover also be acquired by means of a separate magnetic resonance measurement which is designed as separate to the overview measurement and/or to a measurement required for slice positioning. In this case it is possible to exclusively acquire data which comprises information regarding a transition from a tissue to air and thus in particular acquire three-dimensional information regarding an outer contour of the patient 15. Said separate measurement offers a particularly high precision in the acquisition of the outer contour of the patient which means that it is advantageously possible to dispense with further information items and thus measuring time.

In a further method step 101 the first 3D image data acquired by means of the first magnetic resonance measurement is evaluated, wherein the evaluation is performed by the system control unit 21 which to this end includes an evaluation unit (not shown in detail). In method step 101 a three-dimensional spatial arrangement and/or a three-dimensional spatial contour of objects arranged inside the patient receiving area 14, in particular of the patient 15 and where applicable of the motion sensor units 28, are/is determined and/or acquired from the first 3D image data of the first magnetic resonance measurement. Subsequently in a further method step 102 the three-dimensional spatial arrangement and/or a three-dimensional spatial contour of objects arranged inside the patient receiving area 15 are/is stored in a storage unit (not shown in detail) of the system control unit 21.

Concurrently with the acquisition of the first 3D image data first movement data is acquired by means of the motion sensor units 27, 28 in a further method step 103, wherein the movement data comprises 1D movement data and/or 2D movement data. Said first 1D movement data and/or 2D movement data is likewise acquired prior to the medical imaging examination on the patient 15. The first 1D movement data and/or 2D movement data is also evaluated prior to the medical magnetic resonance examination on the patient 15 in a further method step 104. A query 105 subsequently serves to ascertain whether or not the patient 15 has made a movement.

If the patient 15 has moved during the acquisition of the first image data, first movement data is again acquired by means of the motion sensor units 27, 28 and method steps 103, 104, 105 are carried out again.

If the patient 15 has not made any movement during the acquisition of the first image data, the evaluated movement data is likewise stored within the storage unit of the system control unit 21 in a further method step 106.

In a subsequent method step 107 potential movements of the patient 15 are calculated by means of a movement calculator unit 39, wherein both the stored three-dimensional spatial arrangement and/or a three-dimensional spatial contour of the patient 15 arranged inside the patient receiving area 14 and also the stored and evaluated movement data are included in the calculation. The movement calculator unit 39 is designed specifically for calculating potential movements of the patient 14, wherein to this end the movement calculator unit 39 comprises special computer programs and special software units which are executed on a processor (not shown in detail) of the movement calculator unit. In addition, to this end a model calculation and/or a fitting method for calculating a potential movement of the patient 15 are/is also used by the movement calculator unit 39.

In a further method step 108 the potential movements of the patient 15 ascertained on the basis of the three-dimensional spatial arrangement and/or a three-dimensional spatial contour of the patient 15 arranged inside the patient receiving area 14 and the stored and evaluated movement data are subsequently deposited and/or stored in a lookup table such that fast and reliable access to the table is always ensured during the medical imaging examination.

Furthermore, in the event of the patient 15 not moving the magnetic resonance examination is started in a further method step 109 and in the subsequent method step 110 medical 3D image data, in particular medical 3D magnetic resonance data, is recorded and/or acquired. Concurrently with the recording and/or acquisition of medical 3D magnetic resonance data, in a further method step 111 acquisition and/or recording of further movement data also takes place by means of the motion sensor units 27, 28, in which case the further movement data is recorded and/or acquired during the medical magnetic resonance examination. The recording and/or acquisition of the further movement data takes place continuously during the entire medical magnetic resonance examination, which means that current information regarding movements of the patient 15 is always available during the entire magnetic resonance examination. The movement data acquired during the medical magnetic resonance examination is likewise formed from 1D movement data and/or 2D movement data.

The movement data acquired during the medical magnetic resonance examination is evaluated immediately after acquisition in a further method step 112 by the movement calculator unit 39, wherein rapid information regarding a movement is given in this case by means of the data deposited in the lookup table. Furthermore, a plausibility check can be performed within the movement calculator unit 39 on the information regarding movements. To this end for example a plurality of movement data items acquired and/or recorded in temporal succession must lie along a movement trajectory.

Furthermore, in method step 112 a three-dimensional image of the patient and/or of the movement of the patient 15 can also be ascertained by means of the movement calculator unit 39. In this case, 2D movement data is included in the simulation calculation of the movement of the patient 15, such that together with the 3D image data a three-dimensional image of the patient 15 is ascertained. In order to optimize the simulation calculation it is possible in this case by means of a method of least squares and/or further optimization methods to minimize a deviation of the simulated movement using the movement data acquired during the medical imaging examination, as is advantageous in particular in the case of 2D movement data.

A further method step 113 queries whether a movement of the patient 15 has taken place during the medical magnetic resonance examination. If no movement has taken place, a check is subsequently also made in method step 114 as to whether the medical magnetic resonance examination has already finished. If the medical magnetic resonance examination has already finished, the method for determining a movement of the patient 15 during the medical magnetic resonance examination is also terminated. If the medical magnetic resonance examination has not yet finished, 1D movement data and/or 2D movement data is acquired again by means of the motion sensor units 27, 28 in method step 111.

If on the other hand a movement of the patient 15 has taken place during the medical magnetic resonance examination, a further query 115 ascertains whether the movement made by the patient 15 permits a subsequent and/or retrospective correction, as is possible for example in the case of only slight changes in position and/or movements of the patient 15 during the medical magnetic resonance examination. If the movement made by the patient 15 permits a subsequent and/or retrospective correction, in a further method step 116 the 1D movement data and/or 2D movement data acquired by means of the motion sensor units 27, 28 is as a result stored together with the medical 3D image data from the medical magnetic resonance examination. A correction of the movement of the patient 15 during the medical magnetic resonance examination takes place in a further method step 118 only when an evaluation and/or reconstruction of the medical 3D image data occurs, such that in this case artifacts produced due to the movement of the patient 15 during the medical magnetic resonance examination are eliminated in the reconstructed magnetic resonance images. In this case, at least one evaluation parameter for the evaluation and/or reconstruction of the medical 3D image data is modified and/or corrected by the system control unit 21 depending on the information acquired regarding the movement of the patient 15.

A query in method step 117 furthermore ascertains whether the medical magnetic resonance examination has already finished. If the medical magnetic resonance examination has not yet finished, 1D movement data and/or 2D movement data continues to be acquired and further processed in method steps 111-116.

If a subsequent correction is not possible, in a further method step 119 individual measurement parameters of a measurement protocol of the medical magnetic resonance examination are modified and/or corrected by the system control unit 21. In this case, in particular individual parameters for controlling and/or executing individual magnetic resonance sequences are adjusted to the current movement of the patient 15 during the medical magnetic resonance examination. This can for example result in the fact that individual parameters for a magnetic field gradient are adjusted to the movement information regarding the movement of the patient 15 during the medical magnetic resonance examination. Such a modification and/or correction of measurement parameters within the measurement protocol of the medical magnetic resonance examination can moreover result in individual partial measurements of the medical magnetic resonance examination being at least partially aborted, which can result in a resumption of the partial measurements of the medical magnetic resonance examination and/or to a restart of said partial measurements of the medical magnetic resonance examination only when the patient 15 is lying motionless on the patient supporting device 16.

After the adjustment and/or correction of the measurement parameters, a query in a further method step 120 ascertains whether the medical magnetic resonance examination has already finished. If the medical magnetic resonance examination has already finished, the method according to the invention for acquiring a movement of the patient during the medical magnetic resonance examination will also be terminated.

If the medical magnetic resonance examination has not yet finished, 1D movement data and/or 2D movement data continues to be acquired and further processed in method steps 111-119.

It is moreover also conceivable that a movement of the patient can take place in method step 110 and/or in method step 111 by means of marker elements additionally arranged on the patient.

Provision can furthermore also be made that in the method steps 105, 113 in which a movement of the patient is ascertained this is output and/or notified to an operator, for example a member of clinical staff and/or a doctor, as visual information by means of the display unit 22. It is furthermore also conceivable that in method steps 117, 118, 119 a correction and/or modification of parameters, in particular of evaluation parameters and/or of measurement parameters, are/is likewise notified to the operator by means of the display unit 22.

We claim:

1. A method for acquiring a movement of a patient during a medical imaging examination, by means of a medical imaging device, comprising:
acquiring 3D image data by means of the medical imaging device prior to the medical imaging examination;
calculating 3D position information for the patient on the basis of the 3D image data;
acquiring movement data by means of a motion sensor unit during the medical imaging examination; and
determining information regarding the movement of the patient,
wherein the 3D position information for the patient and the movement data acquired by means of the motion sensor unit are included in the calculation.

2. The method as claimed in claim 1,
wherein concurrently with the acquisition of the 3D image data first movement data is acquired by means of the motion sensor unit prior to the medical imaging examination.

3. The method as claimed in claim 2,
wherein from the first movement data acquired prior to the medical imaging examination first information regarding the movement is ascertained which constitutes a start criterion for the medical imaging examination.

4. The method as claimed in claim 3,
wherein the start criterion for the medical imaging examination consists of the patient being motionless.

5. The method as claimed in claim 1,
wherein the movement data acquired during the medical imaging examination is acquired continuously for the duration of the medical imaging examination.

6. The method as claimed in claim 1,
wherein the movement data acquired during the medical imaging examination and information calculated therefrom regarding a movement are stored.

7. The method as claimed in claim 1,
wherein the movement data acquired during the medical imaging examination or information calculated therefrom regarding a movement is stored.

8. The method as claimed in claim 1,
wherein on the basis of information regarding the movement of the patient ascertained from the movement data acquired during the medical imaging device a correction step is started.

9. The method as claimed in claim 8,
wherein the correction step comprises a correction and/or modification of an evaluation parameter during an evaluation of the medical image data from the medical imaging examination.

10. The method as claimed in claims 8,
wherein the correction step comprises a correction and/or modification of a measurement parameter of a measurement protocol of the medical imaging examination.

11. The method as claimed in claim 1,
wherein information regarding a potential movement is calculated on the basis of a simulation of a movement of the patient by means of 3D image data acquired prior to the medical imaging examination and the first movement data acquired prior to the medical imaging examination, and
wherein the simulation at least partially includes a model calculation and a fitting method.

12. The method as claimed in claim 1,
wherein information regarding a potential movement is calculated on the basis of a simulation of a movement of the patient by means of 3D image data acquired prior to the medical imaging examination and the first movement data acquired prior to the medical imaging examination, and
wherein the simulation at least partially includes a model calculation or a fitting method.

13. The method as claimed in claim 12,
wherein a 3D image of the movement of the patient is simulated by means of the simulation.

14. The method as claimed in claim 12,
wherein the potential movement information ascertained comprises a plurality of possible movements which are deposited in a lookup table.

15. The method as claimed in claim 12,
wherein an association and/or a selection of one of the plurality of potential movement information items takes place by means of the movement data acquired during the medical imaging examination.

16. The method as claimed in claim 1,
wherein the motion sensor unit comprises a 1D motion sensor element and a 2D motion sensor element.

17. The method as claimed in claim 1,
wherein the motion sensor unit comprises a 1D motion sensor element or a 2D motion sensor element.

18. The method as claimed in one of the preceding claim 1,
wherein the acquiring of the 3D image data acquired by means of the medical imaging device includes a navigator measurement and/or a measurement required for slice positioning.

19. A medical imaging device, comprising:
a detector unit;
a patient supporting device;
a patient receiving area enclosed by the detector unit, which patient receiving area is designed to accommodate a patient positioned on the patient supporting device, and at least one motion sensor unit; and
a movement calculator unit which is designed to calculate a movement of the patient on the basis of data from the detector unit and on the basis of data from the motion sensor unit as claimed in the method of claim 1.

20. The medical imaging device as claimed in claim 16,
wherein the motion sensor unit comprises at least two motion sensor elements, and
wherein the at least two motion sensor elements each have a 1D motion sensor element or a 2D motion sensor element.
